(19) United States
(12) Patent Application Publication
(10) Pub. No.: US 2008/0187518 A1
(43) Pub. Date: Aug. 7, 2008

(54) Production of Osteoclasts from Adipose Tissues

(75) Inventors: Roy Clinton Ogle, Earlysville, VA (US); Ashok Tholpady, Charlottesville, VA (US); Sunil Shantigodu Tholpady, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 11/915,032

(22) PCT Filed: May 25, 2006
(86) PCT No.: PCT/US2006/020535

§ 371 Date: Nov. 20, 2007

Related U.S. Application Data
(60) Provisional application No. 60/684,599, filed on May 25, 2005.

(51) Int. Cl. A61K 35/12; C12N 5/06; C12Q 1/06; A61P 43/00

(52) U.S. Cl. 424/93.7; 435/325; 435/366; 435/377; 435/378

(57) ABSTRACT

The present invention provides methods for growing and inducing osteoclastic differentiation of adipose tissue derived cells. The present invention further provides methods for administering such adipose tissue derived cells to a subject. The present invention further provides methods for treating diseases, disorders, and conditions associated with aberrant regulation or numbers of osteoclasts.

### CROSS REFERENCE TO RELATED APPLICATIONS

This application claims priority under 35 U.S.C. §119(e) to U.S. Provisional Application Ser. No. 60/684,599 filed May 25, 2005, the disclosure of which is incorporated by reference in its entirety herein.

### STATEMENT REGARDING FEDERALLY SPONSORED RESEARCH OR DEVELOPMENT

This invention was made with United States Government support under National Institutes of Health Grant No. 5R21DE015023-02. The United States Government may therefore have certain rights in the invention.

### BACKGROUND

Stem cells from adipose tissue are a recently discovered reserve of multipotent cells within the body. Adipose stem cells (ASCs) have been shown to have the capacity to differentiate into osteoblasts, myoblasts, adipocytes, chondrocytes, neuron-like cells, and other cell types. Human and animal adipose tissues are abundant and replenishable sources of multipotent stem cells (Zuk et al., Tissue Eng., 2001, 7 (2):211-228). These cells, which can be derived from several distinct adipose tissue sources, are able to proliferate robustly in vitro prior to induction of differentiation to specialized cell types.

It was recently determined that approximately 1-2% of the ASCs grown in tissue culture on polystyrene express markers of the precursors of the monocyte-macrophage lineage (Katz et al., Stem Cells, 2005, 23 (3):412-423). Katz also describes a subpopulation of ASCs that express the osteoclast markers CD11b and 11c. No one has demonstrated that monocytes, macrophages, or related cells can be produced from the CD11b/CD11c positive or negative subpopulations of ASCs.

Adipose tissue offers a potential alternative to the bone marrow as a source of multipotential stem cells. Adipose tissue is readily accessible and abundant in many individuals. Obesity is a condition of epidemic proportions in the United States, where over 50% of adults exceed the recommended BMI based on their height. Adipocytes can be harvested by liposuction on an outpatient basis. This is a relatively non-invasive procedure with cosmetic effects, which are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stem cells capable of self-renewal.

Pathologic evidence suggests that adipose-derived cells are capable of differentiation along multiple mesenchymal lineages. The most common soft tissue tumor, liposarcoma, develops from adipocyte-like cells. Soft tissue tumors of mixed origin are relatively common. These may include elements of adipose tissue, muscle (smooth or skeletal), cartilage, and/or bone. Just as bone-forming cells within the bone marrow can differentiate into adipocytes or fat cells, the extramedullary adipocytes are capable of forming bone. In patients with a rare condition known as paroxysmal osseous heteroplasia, subcutaneous adipocytes form bone for unknown reasons (Kaplan, 1996, Arch. Dermatol. 132:815-818).

Adipose tissue-derived cells represent a stem cell source that can be harvested routinely with minimal risk to the subject. They can be expanded ex vivo, differentiated along unique mesodermal lineage pathways, genetically engineered, and re-introduced into individuals as either an autologous or allogeneic transplantation. This invention presents examples of methods and compositions for the isolation, characterization, and differentiation of adipose tissue-derived cells and outlines their use for the treatment of a number of conditions and diseases.

Osteoclasts, the sole bone-resorbing cells, arise by fusion and differentiation of monocyte/macrophage precursors. Matrix degradation requires adhesion of the osteoclast to bone, an integrin-mediated event that also stimulates signals which polarize the cell and secrete resorptive molecules such as hydrochloric acid and acidic proteases. At least two cytokines are involved in osteoclastogenesis, namely, receptor activator of nuclear factor kappaB ligand (RANKL) and macrophage colony-stimulating factor (M-CSF). Both are produced by mesenchymal cells in the bone marrow environment. M-CSF promotes survival and proliferation of osteoclast precursors. It also contributes to their differentiation and regulates the cytoskeletal changes that accompany bone resorption.

Rapid progress has been made in recent years regarding the mechanisms regulating the formation, activation, and survival of osteoclasts, which are derived from precursor cells in the myeloid lineage. In contrast, study of the regulation of osteoclast precursors (OCPs) has been relatively slow, in part because it has been hard to accurately identify them. However, following the discovery of cell-surface markers that facilitated purification of OCPs, recent studies have demonstrated that peripheral blood OCP numbers are increased in tumor necrosis factor (TNF)-mediated arthritis, both in animals and humans, and these numbers correlate with serum TNF levels. The increase can be reversed by anti-TNF therapy. Furthermore, the precursor cells that give rise to osteoclasts can also differentiate into other cell types, including dendritic cells. Receptor activator nuclear factor-kappaB ligand (RANKL) stimulates OCPs to produce pro-inflammatory cytokines and chemokines, and RANKL blockade prevents joint inflammation in a murine model of inflammatory arthritis. These findings suggest that OCPs may serve as a source for both osteoclasts and other effector cells and participate actively in the pathogenesis of diseases.

Osteoclasts are essential for skeletal development and remodeling throughout the life of animal and man. Deficiency of osteoclasts leads to osteopetrosis, a disease manifested by increased non-remodeled bone mass, which ultimately leads to bone deformities and functional failure of other body systems. The osteopetroses are a heterogeneous group of skeletal disorders characterized by a generalized increase in bone mass caused by decreased bone resorption. On the other hand, increased number and activity of osteoclasts under certain pathologic conditions causes accelerated bone resorption and may lead to osteoporosis and osteolytic diseases. Other processes also involve osteoclasts. Tooth eruption depends on the presence of osteoclasts to create an eruption pathway through the alveolar bone. In diseases where osteoclast formation or function is reduced, such as the various types of osteopetrosis, tooth eruption is affected. Diseases in which osteoclast formation or activity is increased, such as familial expansile osteolysis and Paget's disease, are associated with dental abnormalities such as root resorption and premature tooth loss.

There is a long felt need in the art for methods of identifying sources of osteoclast precursors and osteoclasts which can be used to treat subjects who need such cells. The present invention satisfies this need.

### SUMMARY OF THE INVENTION

It is an object of the invention to provide compositions derived from embryonic, juvenile, or adult adipose tissue, which can be readily obtained and used for the generation of osteoclast precursors and osteoclasts. The invention provides cells that are harvested from lipoaspirates obtained during liposuction procedures such as during abdominoplasty. The invention also encompasses other methods for obtaining cells derived from adipose tissue, such as obtaining cells from reduction mammoplasty procedures. The methodologies and cells described herein can be used for a variety of applications. The osteoclasts, osteoclast precursor cells, and adipose stem cells capable of being induced to differentiate into osteoclasts derived herein can be used, inter alia, to study osteoclast biology (development, commitment, differentiation, metabolism, and cell death) which is normally difficult because of the difficulty of isolating and maintaining these cells. Differentiated osteoclasts of the invention, can also be used for determining the efficacy of drugs that affect their formation, function, and metabolism, in various diseases, disorders, and conditions, including, but not limited to, osteoporosis, osteopetrosis, osteolytic or osteoblastic cancers, and bone trauma, such as fractures.

The present invention provides methods for enrichment and fractionation of adipose cells and methods to induce differentiation of osteoclasts in vitro from a heterogeneous population of cells isolated from adipose tissue. The adipose tissue can be used to generate osteoclast precursor cells in vitro and can be used as a source to deliver such cells in vivo. Thus, the invention provides a new medical use of adipose tissue for the preparation of compositions for augmenting, treating, or altering a subject's bone disease, disorder, condition, or injury. A method for obtaining and propagating osteoclast precursors from mammalian adipose tissue comprises obtaining adipose tissue and placing the adipose tissue progeny in an environment, such as tissue culture or a subject's bone or other tissue, that induces the adipose cells or osteoclast precursors to differentiate into osteoclasts.

The present invention provides a novel method for inducing differentiation of adipose tissue-derived cells into osteoclasts in vitro, as well as methods of local delivery of undifferentiated adipose tissue-derived cells implanted into bone defects, which are induced to become osteoclasts in vivo. The invention provides methods for induction of differentiation in vitro and delivery and differentiation in vivo of osteoclasts from adipose tissue-derived cells.

### BRIEF SUMMARY OF THE DRAWINGS

FIG. 1, comprising FIGS. 1A, 1B, 1C, 1D, and 1E, represents photographic images depicting adipose tissue-derived cells-cultured under various conditions. Adipose tissue-derived cells in an undifferentiated state in serum-containing medium are demonstrated in FIG. 1A and adipose-derived cells differentiated to osteoclasts in vitro in serum-containing medium are demonstrated in FIG. 1B. Some adipose tissue-derived cells were cultured and differentiated in serum-free medium (FIGS. 1C, 1D, and 1E). FIG. 1C represents an image of high density adipose tissue-derived cells induced to differentiate in serum-free conditions, where a high percentage of cells are positive for TRAP. The cells in FIG. 1D were cultured in the presence of RANKL and the cell density is not as high as in FIG. 1C, while the cells in FIG. 1E were cultured in the absence of RANKL. Cells were stained for the presence of tartrate-resistant acid phosphatase (TRAP, dark brown reaction product when viewed in color), a specific marker which, along with large multinucleated nuceli, confirms the osteoclast phenotype. In FIG. 1E, the bar indicator indicates 10 microns.

FIG. 2, comprising FIGS. 2A, 2B, 2C, 2D, 2E, 2F, and 2G, represents photographic images and graphic illustrations (FIG. 2F) depicting histology of regenerating cranial bone tissue following in vivo implantation of pretreated human adipose tissue-derived cells into NIH-Nude rats. The images of 2A and 2B demonstrate the presence of multinucleated osteoclasts within resorption lacunae in the bone. The ASCs are dark (brown), staining positively with a human specific mitochondrial antibody, indicating that the osteoclasts are derived from the implanted human cells rather than from the rat host. FIGS. 2C and 2D are low magnifications of the bone forming in the defect from a group treated with differentiated ASCs depicted in the histogram of FIG. 2F. In FIG. 2C the tissue showed areas containing osteoclasts (at higher magnification in inset). The osteoclast in the inset of FIG. 2C is within a resorption lacuna. Human cells were stained with antibody specific for human mitochondria (magnification bar=50 μM), which results in a dark stain. FIG. 2E represents 3D CT reconstructions of nude rats treated with ASCs in duragen collagen sponges in 8 mm defects. FIG. 2F represents a histogram of percentage of healing of an osseous defect (8 mm) in NIH-Nude rats (including the animals depicted in the X-rays images in right panel). In these experimental groups, 1×10⁶ ASCs from three preparations were delivered in rat collagen I gels, either with no differentiation treatment (U) or were pretreated 10 days with osteoblast-inducing agents to induce osseous differentiation. FIG. 2G represents an image depicting contact planar X-ray images of cranial bone containing excised defects treated for 6 weeks with human skin fibroblasts (top panel), undifferentiated ASCs (middle panel), and osteoblast differentiated (10 days) ASCs delivered in collagen gels to the bone defect.

### DETAILED DESCRIPTION OF THE INVENTION

### Abbreviations, Acronyms, And Definitions

adipose tissue-derived cells (ADCs)

adipose stem cells (ASCs)

calf skin collagen (CSC)

carboxy propeptide of type I collagen (ICTP)

cathepsin K (CTSK)

Dulbecco's modified Eagle medium (DMEM)

Fetal bovine serum (FBS)

fluorescent activated cell sorting (FACS)

macrophage colony stimulating factor (M-CSF)

osteoprotegerin (OPG)

polylactic acid glycolic acid copolymers (PLAGA)

pyridinoline (PYR)

receptor activator of nuclear factor kappa B (RANK)

RANK ligand (receptor activator of nuclear factor kappa B ligand; “RANKL”)

serum-free medium (SFM)

tartrate-resistant acid phophatase (TRAP)

type I collagen cross-linked C-telopeptides (CTX)

type I collagen cross-linked N-telopeptides (NTX)

urinary deoxypyridinoline (D-PYR)

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “aberrant osteoclast activity”, as used herein, refers to not only known functions of osteoclasts which are not occurring at a normal level, but also to numbers of osteoclasts.

By the term “associated with aberrant osteoclast activity” is meant a disease, disorder, or injury which directly or indirectly induces aberrant osteoclast activity or which is manifested following aberrant osteoclast activity.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably, the adipose tissue is human. A convenient source of adipose tissue is from liposuction surgery or procedures such as reduction mammoplasty, however, the source of adipose tissue or the method of isolation of adipose tissue is not critical to the invention.

The term “adipose stem cell,” as used herein, refers to a cell derived from adipose tissue that has the ability to differentiate into at least one differentiated cell type other than into an adipocyte. The phrases “adipose stem cell” and “adipose tissue-derived stem cell” are used interchangeably herein.

The term “adult” as used herein, is meant to refer to any non-embryonic or non-juvenile animal. For example the term “adult adipose tissue stem cell,” refers to an adipose stem cell, other than that obtained from an embryo or juvenile animal.

As used herein, the term “affected cell” refers to a cell of a subject afflicted with a disease or disorder, which affected cell has an altered phenotype relative to a subject not afflicted with a disease or disorder.

Cells or tissue are “affected” by a disease or disorder if the cells or tissue have an altered phenotype relative to the same cells or tissue in a subject not afflicted with a disease or disorder.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder. A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

The term “biocompatible,” as used herein, refers to a material that does not elicit a substantial detrimental response in the host.

The terms “cell” and “cell line,” as used herein, may be used interchangeably. All of these terms also include their progeny, which are any and all subsequent generations. It is understood that all progeny may not be identical due to deliberate or inadvertent mutations.

The terms “cell culture” and “culture,” as used herein, refer to the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.”

The phrases “cell culture medium,” “culture medium” (plural “media” in each case) and “medium formulation” refer to a nutritive solution for cultivating cells and may be used interchangeably.

A “compound,” as used herein, refers to a polypeptide, an isolated nucleic acid, and to any type of substance or agent that is commonly considered a chemical, drug, or a candidate for use as a drug, as well as combinations and mixtures of the above.

A “conditioned medium” is one prepared by culturing a first population of cells or tissue in a medium, and then harvesting the medium. The conditioned medium (along with anything secreted into the medium by the cells) may then be used to support the growth or differentiation of a second population of cells.

The term “delivery vehicle” refers to any kind of device or material which can be used to deliver cells in vivo or can be added to a composition comprising cells administered to an animal. This includes, but is not limited to, implantable devices, matrix materials, gels, etc.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

A “disease or disorder associated with aberrant osteoclast activity” refers to a disease or disorder comprising either increased or decreased: osteoclast activity; numbers of osteoclasts; or numbers of osteoclast precursors.

As used herein, an “effective amount” means an amount of a compound or agent sufficient to produce a selected or desired effect. The term “effective amount” is used interchangeably with “effective concentration” herein.

The term “enhancing bone repair” as used herein refers to methods of speeding up or inducing better bone repair using the methods of the invention, relative to the speed or amount of bone repair that occurs without practicing the methods of the invention.

The term “feeder cells” as used herein refers to cells of one type that are co-cultured with cells of a second type, to provide an environment in which the cells of the second type can be maintained, and perhaps proliferate. The feeder cells can be from a different species than the cells they are supporting. The terms, “feeder cells”, “feeders,” and “feeder layers” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Graft” refers to any free (unattached) cell, tissue, or organ for transplantation.

“Allograft” refers to a transplanted cell, tissue, or organ derived from a different animal of the same species.

“Xenograft” refers to a transplanted cell, tissue, or organ derived from an animal of a different species.

The term “ingredient” refers to any compound, whether of chemical or biological origin, that can be used in cell culture media to maintain or promote the growth or proliferation of cells. The terms “component,” “nutrient” and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

The term “inhibit,” as used herein, means to suppress or block an activity or function such that it is lower relative to a control value. The inhibition can be via direct or indirect mechanisms. In one aspect, the activity is suppressed or blocked by at least 10% compared to a control value, more preferably by at least 25%, and even more preferably by at least 50%. The term “inhibitor” as used herein, refers to any compound or agent, the application of which results in the inhibition of a process or function of interest, including, but not limited to, differentiation and activity. Inhibition can be inferred if there is a reduction in the activity or function of interest.

The term “injury” refers to any physical damage to the body caused by violence, accident, trauma, or fracture, etc.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

As used herein, the term “insult” refers to injury, disease, or contact with a substance or environmental change that results in an alteration of tissue or normal cellular metabolism in a tissue, cell, or population of cells.

The term “isolated,” used in reference to a single cell of interest or population of cells of interest at least partially isolated other cell types or other cellular material with which it naturally occurs in the tissue of origin (e.g., adipose tissue). A sample of stem cells is “substantially pure” when it is at least 60%, or at least 75%, or at least 90%, and, in certain cases, at least 99% free of cells other than cells of interest. Purity can be measured by any appropriate method, for example, by fluorescence-activated cell sorting (FACS), or other assays which distinguish cell types.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

The term “modulate”, as used herein, refers to changing the level of an activity, function, or process. The term “modulate” encompasses both inhibiting and stimulating an activity, function, or process.

The phrase “osteoclast-associated disease” as used herein refers to any disease, disorder or condition wherein the disease, disorder or condition is mediated by osteoclasts or associated with, but not limited to, changes in osteoclast activity, function, numbers of osteoclasts, and numbers of osteoclast precursors, and growth or differentiation of osteoclast precursors.

As used herein, the phrase “osteoclast differentiation-inducing factor” means one or more factors capable of inducing osteoclast differentiation. The factors may be known factors which can be added to cells in culture or administered to a subject or may be factors which are present in vivo and can induce osteoclast differentiation.

As used herein, an “osteoclast precursor cell” is any cell capable of differentiating or maturing into a mature osteoclast, wherein a mature osteoclast is a cell capable of resorbing bone in vitro or in vivo.

The phrase “osteoclast production”, as used herein, refers to the processes involved in the formation, proliferation, differentiation, maturation, activation, and survival of osteoclasts.

“Osteogenesis” as used herein refers to bone growth, bone remodeling, and repair of bone due to injury or disease.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “purified” and like terms relate to an enrichment of a cell, cell type, molecule, or compound relative to other components normally associated with the cell, cell type, molecule, or compound in a native environment. The term “purified” does not necessarily indicate that complete purity of the particular cell, cell type, molecule, or compound has been achieved during the process.

A “reversibly implantable” device is one which may be inserted (e.g. surgically or by insertion into a natural orifice of the animal) into the body of an animal and thereafter removed without great harm to the health of the animal.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In one aspect, the activity or differentiation is stimulated by at least 10% compared to a control value, more preferably by at least 25%, and even more preferably by at least 50%. The term “stimulator” as used herein, refers to any compound or agent, the application of which results in the stimulation of a process or function of interest, including, but not limited to, osteoclast production, differentiation, and activity.

A “subject” of analysis, diagnosis, or treatment is an animal. Such animals include mammals, preferably a human.

The term “substantially pure” describes a compound, e.g., a protein or polypeptide or other compound which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, the term “treating” includes prophylaxis of a specific disease, disorder, or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “wound” relates to a physical tear or rupture to a tissue or cell layer. A wound may occur by any physical insult, including a surgical procedure.

The present invention lies in the significant discovery that adipose tissue contains osteoclast precursor cells that can be induced to simply and efficiently produce osteoclasts in vitro and in vivo. The present invention provides methods and compositions for the culture of adipose tissue-derived cells and their differentiation into osteoclast lineage cells.

The cells produced by the methods of invention are useful in providing a source of adipose tissue-derived cells with the ability to differentiate into osteoclasts, as well as of fully differentiated and functional cells for research, transplantation, and development of tissue engineering products for the treatment of diseases and disorders and traumatic injury repair. Thus, in one aspect, the invention provides a method for differentiating adipose tissue-derived cells along the osteoclast lineage and into functional osteoclasts.

In one embodiment of the invention, enriched populations can be used for tissue engineering applications in which bone needs to be remodeled or removed, or drugs need to be delivered to bone. The invention encompasses various methods for enriching populations of adipose tissue-derived cells from adipose tissue and propagating the cells in culture. The present invention further provides compositions and methods for growing adipose tissue-derived cells in serum-free medium and for inducing osteoclast differentiation of adipose stem cells in serum-free medium. It is also demonstrated herein that administration of cultured adipose tissue-derived cells to a subject results in bone formation comprising osteoclasts which arose from the implanted cells.

The adipose tissue-derived cells useful in the methods of invention can be isolated by a variety of methods known to those skilled in the art such as described in WO 00/53795. In a preferred method, adipose tissue is isolated from a mammalian subject, preferably a human subject. A preferred source of adipose is subcutaneous adipose tissue. Another preferred source of adipose tissue is omental adipose. In humans, the adipose is typically isolated by liposuction. If the cells of the invention are to be transplanted into a human subject, it is preferable that the adipose tissue be isolated from that same subject to provide for an autologous transplant. Alternatively, the transplanted cells are allogeneic.

Adipose tissue-derived cells represent a stem cell source that can be harvested routinely with minimal risk or discomfort to the subject. They can be expanded ex vivo, differentiated along unique lineage pathways, genetically engineered, and re-introduced into individuals as either autologous or allogeneic transplantation.

Other methods for the isolation, expansion, and differentiation of human adipose tissue-derived cells have been reported. See for example, Burris et al., 1999, Mol. Endocrinol. 13:410-7; Erickson et al., 2002, Biochem. Biophys. Res. Commun., 2002, 290 (2):763-9; Gronthos et al., 2001, J. Cell. Physiol., 189:54-63; Halvorsen et al., 2001, Metabolism 50:407-413; Halvorsen et al., 2001, Tissue Eng. 7 (6):729-41; Harp et al., 2001, Biochem. Biophys. Res. Commun 281:907-912; Saladin et al., 1999, Cell Growth & Diff 10:43-48; Sen et al., 2001, J. Cell. Biochem. 81:312-319; Zhou et al., 1999, Biotechnol. Techniques 13:513-517. Adipose tissue-derived cells (also referred to as “adipose-derived cells” or “ADCs”) are obtained from minced human adipose tissue by collagenase digestion and differential centrifugation (Halvorsen et al., 2001, Metabolism 50:407-413; Hauner et al., 1989, J. Clin. Invest. 84:1663-1670; Rodbell et al., 1966, J. Biol. Chem. 241:130-139). Others have demonstrated that human adipose tissue-derived cells can be cultured and induced to differentiate along the adipocyte, chondrocyte, and osteoblast lineage pathways (Erickson et al., 2002, Biochem. Biophys. Res. Commun., 2002; 290 (2):763-9; Gronthos et al., 2001, J. Cell. Physiol., 189:54-63; Halvorsen et al., 2001, Metabolism 50:407-413; Halvorsen et al, 2001, Tissue Eng., 2001; (6):729-41; Harp et al., 2001, Biochem. Biophys. Res. Commun. 281:907-912; Saladin et al., 1999, Cell Growth & Diff. 10:43-48; Sen et al., 2001, J. Cell. Biochem. 81:312-319; Zhou et al., 1999, Biotechnol. Techniques 13:513-517; Zuk et al., 2001, Tissue Eng. 7: 211-228).

U.S. Pat. No. 6,555,374 (Gimble et al.) claims “[a] non-naturally-occurring mixture of cells comprising an isolated extramedullary adipose tissue-derived stromal cell and a non-adipose derived cell capable of forming a blood cell,” (claim 1) and further, “wherein the formed blood cell is an osteoclast” (claim 15). Therefore, formation of osteoclasts using this claimed mixture requires a non-adipose derived cell. It has not been previously reported that ASC can differentiate into osteoclasts (i.e., that ASCs are comprised of osteoclast precursor cells) or that osteoclast precursor cells exist within adipose tissue.

Lendeckel et al. disclosed the use of adipose tissue to augment cancellous bone reconstruction (J. Craniomaxillofac. Surg., 2004, 32:6:370-373).

In general, cell culture media provide the nutrients necessary to maintain and grow cells in a controlled, artificial, and in vitro environment. Characteristics and compositions of cell culture media vary depending on the particular cellular requirements. Important parameters include osmolarity, pH, and nutrient formulations.

Typically, cell culture medium formulations are supplemented with a range of additives, including undefined components such as fetal bovine serum (FBS) (5-20% v/v) or extracts from animal embryos, organs or glands (0.5-10% v/v). While FBS is the most commonly applied supplement in animal cell culture media, other serum sources are also routinely used, including newborn calf, horse, and human. Organs or glands that have been used to prepare extracts for the supplementation of culture media include submaxillary gland (Cohen (1961) J. Biol. Chem. 237: 1555-1565), pituitary (Peehl and Ham (1980) In Vitro 16: 516-525; see U.S. Pat. No. 4,673,649), hypothalamus, (Maciag, et al. (1979) Proc. Natl. Acad. Sci. USA 76: 5674-5678; Gilchrest, et al. (1984) J. Cell. Physiol. 120: 377-383), and brain (Maciag, et al. (1981) Science 211: 1452-1454). These types of chemically undefined supplements serve several useful functions in cell culture media (see Lambert, et al. (1985) In: Animal Cell Biotechnology, Vol. 1, Spier et al., Eds., Academic Press, New York, pp. 85-122 (1985)). For example, these supplements (1) provide carriers or chelators for labile or water-insoluble nutrients; (2) bind and neutralize toxic moieties; (3) provide hormones and growth factors, protease inhibitors and essential, often unidentified or undefined low molecular weight nutrients; and (4) protect cells from physical stress and damage. Thus, serum or organ/gland extracts are commonly used as relatively low-cost supplements to provide an optimal culture medium for the cultivation of animal cells. “Non-peptide growth factors” refers to non-peptide compounds such as steroids, retinoids and other chemical compounds or agents which regulate cell growth and differentiation. It is generally recognized that concentrations may vary.

The present invention provides serum-free culture conditions for growing adipose tissue-derived cells and for inducing osteoclast differentiation of such cells. The present invention discloses that at least two different commercially available serum-free medium formulations can be used to serially propagate adipose tissue-derived cells and can be used as the base medium for inducing osteoclast differentiation of adipose tissue-derived cells. In one aspect, the invention provides pretreating the tissue culture dishes with factors which enhance cell adhesion and attachment. In one embodiment, one or more extracellular matrix proteins or other molecules can be applied to the tissue culture surface to enhance adhesion of the cells to the dish. In one aspect, the protein is collagen. In a further aspect, the collagen is calf skin collagen. In yet a further aspect, the collagen is collagen type I.

A number of so-called “defined” media, which avoid the use of animal serum (and/or animal extracts), have also been developed. These media, which often are specifically formulated to support the culture of a single cell type, contain no undefined supplements and instead incorporate defined quantities of purified growth factors, proteins, lipoproteins and other substances usually provided by the serum or extract supplement. Because the components (and concentrations thereof) in such culture media are precisely known, these media are generally referred to as “defined culture media.” Often used interchangeably with “defined culture medium” is the term “serum-free medium” or “SFM.” A number of SFM formulations are commercially available, such as those designed to support the culture of endothelial cells, keratinocytes, melanocytes, mammary epithelial cells, monocytes/macrophages, fibroblasts, chondrocytes, and hepatocytes. The distinction between SFM and defined media, however, is that SFM are media devoid of serum, but not necessarily of other undefined components such as organ/gland extracts.

Some extremely simple defined media, which consist essentially of vitamins, amino acids, organic and inorganic salts and buffers, have been used for cell culture. Such media (often called “basal media”) are often deficient in the nutritional content required by most animal cells and may incorporate into the basal medium additional components to make the medium more nutritionally complex, but to maintain the serum-free and low protein content of the media. Non-limiting examples of such components include serum albumin from bovine (BSA) or human (HSA); certain growth factors derived from natural (animal) or recombinant sources; lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements (reviewed by Waymouth (1984) In: Cell Culture Methods for Molecular and Cell Biology, Vol. 1: Methods for Preparation of Media, Supplements, and Substrata for Serum-Free Animal Cell Culture, Barnes et al., eds., New York: Alan R. Liss, Inc., pp. 23-68; and by Gospodarowicz, Id., at pp 69-86).

The advantages of using serum-free media and defined media for drug screening and generation of cellular products for clinical use are well-known in the art. These advantages include, but are not limited to, absence of adventitious organisms such as animal retroviruses, greater control of batch-to-batch variability, and defined levels of known modulators of cell function and activity.

One of ordinary skill in the art will appreciate that culture conditions such as cell seeding densities can be selected for each experimental condition or intended use.

Many techniques are known to those of skill in the art for measuring osteoclast differentiation and those not described herein are encompassed within the techniques of the invention.

Cell culture models for various disorders are useful, e.g., for testing the ability of a compound to modulate a cellular process associated with the disorder. The adipose tissue-derived cells described herein are useful, e.g., for providing a pool of cells that can be differentiated at will and used in assays of such compounds.

The invention further provides for methods of using such cells in toxicological, carcinogen, and drug screening methods, as well as in therapeutic applications where bone and cellular function is replaced or otherwise supplanted using such cells.

In one embodiment, adipose tissue, or cells derived from adipose tissue, are subjected to varied concentrations of osteoclast differentiation-inducing agents to induce osteoclast differentiation. Such agents include, but are not limited to, macrophage colony stimulating factor (M-CSF), RANK ligand (RANKL), and steroids (such as dexamethasone). In one aspect, M-CSF can be used at concentrations ranging from about 1.0 ng/ml to about 300 ng/ml, more preferably from about 10 ng/ml to about 150 ng/ml. In another aspect, RANKL can be used at a concentration ranging from about 1.0 ng/ml to about 1,000 ng/ml, more preferably from about 10 ng/ml to about 200 ng/ml. In yet another aspect, dexamethasone can be used at a concentration ranging from about 1×10⁻⁸ M to about 1×10⁻⁶ M, more preferably from about 5×10⁻⁸ M to about 5×10⁻⁷ M. One of ordinary skill in the art will appreciate that the amount of differentiation-inducing agent(s) used may vary according to the culture conditions, amount of additional differentiation-inducing agent used, or the number of combination of differentiation-inducing agents used when more than one agent is used to induce osteoclast differentiation. Additionally, any compound or agent that activates NFkB or its pathway can be used to help induce osteoclast differentiation. Furthermore, any drug or compound that activates or stimulates RANKL or its signal transduction pathway via its receptor can be used for inducing osteoclast differentiation. For example, steroids and other agents which work via similar or identical nuclear receptor pathways to dexamethasone, such as vitamin D3 and retinoic acid, may be used in the method disclosed herein. Other compounds known in the art may also be used to induce osteoclast differentiation. These compounds include IL-3, IL-7, GM-CSF, eotaxin, eotaxin-2, eotaxin-3, TGF-β, and TNF-α.

In one embodiment, osteoclast differentiation can be negatively regulated. In one aspect, osteoclast differentiation factors are not used or are removed. In another aspect, GM-CSF could be used to induce some of the progenitors along the macrophage lineage, thus preventing those precursors from differentiating into osteoclasts. In yet another aspect, osteoprotegerin (OPG) can be used to bind to RANK and block activation by RANKL.

Some examples of diseases that may be treated according to the methods of the invention are discussed herein. The invention should not be construed as being limited solely to these examples, as other osteoclast-associated diseases that are at present unknown, once known, may also be treatable using the methods of the invention.

Other techniques useful for isolating and characterizing the cells described herein include fractionating cells using CD11b, CD11c, CD14, CD51, and CD61.

One of ordinary skill in the art will appreciate that a variety of techniques can be used to measure the differentiation and function of osteoclasts. For example, Ishida et al. demonstrated a variety of genes useful for such an analysis, including nuclear factor of activated T cells (J. Biol. Chem., 2002, 277:43:41147-41156). See also Maeda et al., U.S. Pat. No. 6,861,257, and Helfrich, Archives of Oral Biology, 2005,50:2:115-122. Osteoclast markers include, but are not limited to, TRAP, calcitonin receptor, multinucleation with ruffled border and resorptive vacuoles, CD11b, CD11c, CTSK, and vitronectin receptors. A resorptive osteoclast exhibits a well-defined ring of actin around the nuclear region within the cell. cSRC is also located around this ring inside the cell and the vitronectin receptor (CD51/CD61) is in the same position on the underside of the cell to form a tight seal with bone during the resorptive process. Assays for osteoclast activity include assays such as bone resorption using dentine slices or the use of calcium phosphate sintering quartz discs (see U.S. Pat. No. 6,861,257).

Markers of bone resorption include urinary hydroxyproline, urinary calcium, TRAP, the collagen cross-links urinary pyridinoline (PYR) and urinary deoxypyridinoline (D-PYR), carboxy propeptide of type I collagen (ICTP), type I collagen cross-linked C-telopeptides (CTX), and type I collagen cross-linked N-telopeptides (NTX). CTX and NTX can be measured in serum or urine.

The present invention also encompasses pharmaceutical and therapeutic compositions comprising the adipose tissue-derived stem cells, purified osteoclast precursors, and differentiated osteoclasts of the present invention. In one embodiment of the invention, therapies are provided for diseases, disorders, or conditions associated with aberrant osteoclast function, activity, numbers, or regulation. Because of the ease of isolation of ADCs and abundance of adipose tissue, these methods are superior to others using bone marrow aspirates, stem cells or circulating blood cells to produce osteoclasts. In one aspect, the invention provides administering to a subject in need thereof a pharmaceutical composition comprising a therapeutically effective amount of adipose tissue-derived cells, osteoclast precursors, or osteoclasts derived therefrom. Use of the terms “adipose stem cells” and “osteoclasts” is not intended to limit the cells to a specific step in the proliferation or differentiation process of the cell lineage. For example, an “osteoclast precursor cell” can mean one that is multipotent or one that can only be induced along the osteoclast differentiation pathway.

The present invention provides methods for administering ADCs to subjects in need thereof. In one aspect, the ADCs have been pretreated to differentiate into osteoclasts. In another aspect, the cells have been pretreated to differentiate into osteoblasts. In yet another aspect, populations of ADCs can be treated with more than one type of differentiation inducing agents or medium, or a combination of agents which induce more than one type of differentiation. In another aspect, separate populations of ADCs, that have been pretreated with osteoclast differentiation-inducing compounds, osteoblast differentiation-inducing compounds, or no treatment at all, can be co-administered to a subject. Co-administration of different groups of cells does not necessarily mean that the ADC populations are actually administered at the same time or that the populations are combined for administered in the same composition. The invention further provides compositions and methods for administering ADCs to subjects and then inducing the ADCs to differentiate into osteoclasts by administering osteoclast differentiation-inducing agents to the subject. In one aspect, the subject is a human. When more than one differentiation agent or compound is used to induce cells along the osteoclast pathway, or when additional agents are also used to induce some of the cells to differentiate along a pathway other than osteoclasts, the various agents need not be provided at the same time.

For example, treatment of reductions in osteoclast activity is encompassed within the therapeutic uses of the cells of the invention. These include recessive osteopetrosis, ADO II, dental abnormalities, and Pycnodysostosis. Several gene defects related to these diseases and disorder include defects in the genes TCIRGI, CLCN7, CAII, OSTMI, and CTSK. Osteoporosis is a disorder that occurs when the amount of bone removed from the skeleton by bone-resorbing osteoclasts exceeds the amount of bone formed by osteoblasts.

Subjects in need of removal of bony overgrowth, such as in skeletal hyperplasia, may also benefit from administration of cells of the invention.

In accordance with one embodiment of the invention, a method is provided for regulating tumor growth, angiogenesis, cell adhesion and osteogenesis, as well as diseases and disorders thereof. Subjects suffering from a cancer which alters osteoblast or osteoclast activity can be treated with cells of the invention. Such cancers include, but are not limited to, breast cancer, prostate cancer, renal carcinoma, and multiple myeloma.

The cells of the present invention may be administered to a subject alone or in admixture with a composition useful in the repair of bone wounds and defects. Such compositions include, but are not limited to bone morphogenetic proteins, hydroxyapatite/tricalcium phosphate particles (HA/TCP), gelatin, poly-L-lysine, and collagen.

Non-synthetic matrix proteins like collagen, glycosaminoglyeans, and hyaluronic acid, which are enzymatically digested in the body, are useful for delivery to bone areas (see U.S. Pat. Nos. 4,394,320; 4,472,840; 5,366,509; 5,606,019; 5,645,591; and 5,683,459) and are suitable for use with the present invention. Other implantable media and devices can be used for delivery of the cells of the invention in vivo. These include, but are not limited to, sponges, such as those from Integra, fibrin gels, scaffolds formed from sintered microspheres of polylactic acid glycolic acid copolymers (PLAGA), and nanofibers formed from native collagen, as well as other proteins. The cells of the present invention can be further combined with demineralized bone material, growth factors, nutrient factors, pharmaceuticals, calcium-containing compounds, anti-inflammatory agents, antimicrobial agents, or any other substance capable of expediting or facilitating bone growth.

Examples of osteoinductive factors suitable for use with the compositions of the present invention include demineralized bone particles, a Bone Morphogenetic Protein, an osteoinductive extract of demineralized bone matrix, or a combination thereof.

Examples of growth factors suitable for use with the composition of the present invention include Transforming Growth Factor-Beta (TGF-β), Transforming Growth Factor-Alpha (TGF-α), Epidermal Growth Factor (EGF), Insulin Like Growth Factor-I or II, Interleukin-I (IL-I), Interferon, Tumor Necrosis Factor, Fibroblast Growth Factor (FGF), Platelet Derived Growth Factor (PDGF), M-CSF, and Nerve Growth Factor (NGF).

The compositions of the present invention can also be combined with inorganic fillers or particles. For example for use in implantable grafts the inorganic fillers or particles can be selected from hydroxyapatite, tri-calcium phosphate, ceramic glass, amorphous calcium phosphate, porous ceramic particles or powders, mesh titanium or titanium alloy, or particulate titanium or titanium alloy.

In one embodiment, the method comprises the steps of administering cells of the present invention to a subject in need thereof. In one embodiment, a composition comprising these cells is administered locally by injection. Compositions comprising the cells can be further combined with known drugs, and in one embodiment, the drugs are bound to the cells. These compositions can be prepared in the form of an implantable device that can be molded to a desired shape. In one embodiment, a graft construct is prepared comprising a biocompatible matrix and one or more cells of the present invention, wherein the matrix is formed in a shape to fill a gap or space created by the removal of a tumor, injured, or diseased tissue.

The cells can be seeded onto the desired site within the tissue to establish a population. Cells can be transferred to sites in vivo using devices such as catheters, trocars, cannulae, stents (which can be seeded with the cells), etc.

No other process is known which can deliver such incredibly large numbers of osteoclast precursors or osteoclasts for the procedures and treatments described herein. Additionally, for diseases that require osteoclast infusions (such as osteopetrosis), adipose tissue harvest is minimally invasive, yields many cells, and can be done repeatedly. This is in stark contrast to the standard therapy which uses bone marrow transplants.

The present invention encompasses the preparation and use of immortalized cell lines, including, but not limited to, osteoclast cell lines or adipose tissue-derived cell lines capable of differentiating into osteoclasts. Various techniques for preparing immortalized cell lines are known to those of ordinary skill in the art. The present invention also encompasses the preparation and use of cell lines or cultures for testing or identifying agents for their effects on bone via effects on osteoclast growth, differentiation, and metabolism. In one aspect, the invention encompasses testing the osteolytic potential or activity of various cancers. In another aspect, the invention encompasses a system to screen agents useful for treating osteoclast associated diseases or disorders. The present invention further encompasses compounds which are identified using any of the methods described herein. Such compounds may be formulated and administered to a subject for treatment of the diseases, disorders, or conditions disclosed herein.

In one embodiment, genes of interest can be introduced into cells of the invention. In one aspect, such cells can be administered to a subject. In one aspect, the subject is afflicted with a bony disease, disorder, or condition. In one aspect, the cells are modified to express exogenous genes or are modified to repress the expression of endogenous genes, and the invention provides a method of genetically modifying such cells and populations. In accordance with this method, the cell is exposed to a gene transfer vector comprising a nucleic acid including a transgene, such that the nucleic acid is introduced into the cell under conditions appropriate for the transgene to be expressed within the cell. The transgene generally is an expression cassette, including a coding polynucleotide operably linked to a suitable promoter. The coding polynucleotide can encode a protein, or it can encode biologically active RNA (e.g., antisense RNA or a ribozyme). Thus, for example, the coding polynucleotide can encode a gene conferring resistance to a toxin, a hormone (such as peptide growth hormones, hormone releasing factors, sex hormones, adrenocorticotrophic hormones, cytokines (e.g., interferons, interleukins, lymphokines), etc.), a cell-surface-bound intracellular signaling moiety (e.g., cell adhesion molecules, hormone receptors, etc.), a factor promoting a given lineage of differentiation, etc.

In addition to serving as useful targets for genetic modification, many cells and populations of the present invention secrete various polypeptides. Such cells can be employed as bioreactors to provide a ready source of a given hormone, and the invention pertains to a method of obtaining polypeptides from such cells. In accordance with the method, the cells are cultured under suitable conditions for them to secrete the polypeptide into the culture medium. After a suitable period of time, and preferably periodically, the medium is harvested and processed to isolate the polypeptide from the medium. Any standard method (e.g., gel or affinity chromatography, dialysis, lyophilization, etc.) can be used to purify the hormone from the medium, many of which are known in the art.

In other embodiments, cells (and populations) of the present invention secreting polypeptides can be employed as therapeutic agents. Generally, such methods involve transferring the cells to desired tissue, either in vitro or in vivo, to animal tissue directly. The cells can be transferred to the desired tissue by any method appropriate, which generally will vary according to the tissue type.

Compositions comprising cells of the invention can be employed in any suitable manner to facilitate the growth and differentiation of the desired tissue. For example, the composition can be constructed using three-dimensional or stereotactic modeling techniques. Thus, for example, a layer or domain within the composition can be populated by cells primed for bone formation. To direct the growth and differentiation of the desired structure, the composition can be cultured ex vivo in a bioreactor or incubator, as appropriate. In other embodiments, the structure is implanted within the host animal directly at the site in which it is desired to grow the tissue or structure. In still another embodiment, the composition can be engrafted onto a host, where it will grow and mature until ready for use. Thereafter, the mature structure (or anlage) is excised from the host and implanted into the host, as appropriate.

Matrices suitable for inclusion into the composition can be derived from various sources. As discussed above, the cells, matrices, and compositions of the invention can be used in bone engineering and regeneration. Thus, the invention pertains to an implantable structure (i.e., an implant) incorporating any of these inventive features. The exact nature of the implant will vary according to the intended use. The implant can be, or comprise, as described, mature or immature tissue. Thus, for example, one type of implant can be a bone implant, comprising a population of the inventive cells that are undergoing (or are primed for) osteoclastic differentiation, optionally seeded within a matrix material. Such an implant can be applied or engrafted to encourage the generation or regeneration of mature bone tissue within the subject.

One aspect of the present invention relates to osteogenic devices, and more specifically to synthetic implants which induce osteogenesis in vivo in mammals, including humans. More particularly, this embodiment of the invention relates to biocompatible, bioresorbable, synthetic compositions comprising the cells disclosed herein. The implants can be prepared using previously described implant materials such as hydroxylapatite, autogenous bone grafts, allogenic bone matrix, demineralized bone powder, and collagenous matrix. The compositions and cells of the present invention can be combined with known graft materials that are fully formable at temperatures above 38° C. but become a solid at temperatures below 38° C. In another embodiment, the cells are combined with known materials to provide a composition for coating implantable prosthetic devices, and to increase the cellular ingrowth into such devices.

In one aspect of the invention, matrix material is provided as a coating on an implant placed in contact with viable bone. Useful implants are composed of an inert material such as ceramic, glass, metal, or polymer. In another aspect, bone growth is induced from a viable mammalian bone by contacting the bone with matrix material comprising cells of the present invention, into which has been dispersed a glue in an amount sufficient to solidify the matrix when implanted in a mammal or when placed at 37° C. One glue suitable for such use is methyl cellulose.

One of ordinary skill in the art would appreciate that there are other carriers 30 useful for delivering the cells of the invention. Such carriers include, but are not limited to, calcium phosphate, hydroxyapatite, and synthetic or natural polymers such as collagen or collagen fragments in soluble or aggregated forms. In one aspect, such carriers serve to deliver the cells to a location or to several locations. In another aspect, the carriers and cells can be delivered either through systemic administration or by implantation. Implantation can be into one site or into several sites.

In certain useful applications, compounds are screened specifically for potential toxicity. Cytotoxicity can be determined in the first instance by the effect on cell viability, survival, morphology, and leakage of enzymes into the culture medium. Other methods to evaluate toxicity include determination of the synthesis and secretion of target proteins of interest and induction of apoptosis (indicated by cell rounding, condensation of chromatin, and nuclear fragmentation). DNA synthesis can be measured using assays such as tritiated-thymidine or BrdU incorporation. Effects of a drug on DNA synthesis or structure can be determined by measuring DNA synthesis or repair. Aberrant DNA synthesis, especially at unscheduled times in the cell cycle, or above the level required for cell replication, is consistent with a drug effect. Unwanted effects can also include unusual rates of sister chromatid exchange, determined by metaphase spread (see pp 375-410 of Vickers (1997) In vitro Methods in Pharmaceutical Research Academic Press).

In one embodiment, the cells of the invention are used to screen factors that promote osteoclast differentiation or promote proliferation and maintenance of such cells in long-term culture. In one aspect, osteoclasts are characterized by the expression of calcitonin receptors, large numbers of vitronectin receptors, the expression of cathepsin K (CTSK) and tartrate-resistant acid phosphatase (TRAP), CD11b, CD11c, and multinucleation.

In another embodiment, cells of the invention are used to screen factors that inhibit osteoclast differentiation.

In yet another embodiment, differentiated or undifferentiated cells of the invention are used to screen factors that modulate osteoclast production, differentiation, function, and activity.

In general, methods for the identification of a compound which effects the differentiation, production, activity, or function of a cell of the invention, include the following general steps:

The test compound is administered to a cell, tissue, sample, or subject, in which the measurements are to be taken. A control is a cell, tissue, sample, or subject in which the test compound has not been added. A higher or lower level of the indicator or parameter being tested, i.e., osteoclast number, osteoclast production, differentiation, activity, function, etc., in the presence of the test compound, compared with the levels of the indicator or parameter in the sample which was not treated with the test compound, is an indication that the test compound has an effect on the indicator or parameter being measured, and as such, is a candidate for modulation of the desired activity. Test compounds may be added at varying doses and frequencies to determine the effective amount of the compound which should be used and effective intervals in which it should be administered. In another aspect, a derivative or modification of the test compound may be used.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

The following examples provide methodologies for in vitro differentiation of osteoclasts from cells derived from adipose tissues, including lipoaspirates obtained by liposuction procedures and applications of the cells for in vitro studies. The following examples also provide methodologies for delivery and in vivo differentiation of osteoclasts from cells derived from adipose tissues including lipoaspirates obtained by liposuction procedures for biomedical and tissue engineering applications in vivo.

### General Materials And Methods

Human Adipose Tissue Isolation And Culture of Adipose Tissue-Derived Cells

Human adipose tissues were obtained from elective abdominoplasties or liposuction procedures performed at the University of Virginia Health Sciences Center with appropriate institutional review board approval and patient consent documentation. Adipose tissue was mechanically isolated from abdominoplasty tissue discards with lipoaspiration and washed extensively with phosphate buffered saline and centrifuged at 2050×g for 5 minutes. After transferring the pelleted cell fraction to another container, Blendzyme was added and the mixture was incubated for 45 minutes at 37° C. under constant agitation. The treated material was centrifuged at 2050×g for 5 minutes; the supernatant fraction was filtered through 250 μm mesh. This filtrate was centrifuged at 2050×g for 5 minutes and the cell pellet was resuspended in erythrocyte lysis buffer for 5 minutes. After centrifuging the mixture at 2050×g for 5 minutes, the supernatant fraction was resuspended in 10% FBS/DMEM/F12 and plated in 10-cm diameter culture dishes at a density of 1×10⁶ cells per plate. Cells were fed every other day by replacing spent media with fresh media.

The cells used in this invention may be the total cell population from culture on plastic dishes (enriched in ASCs), cells freshly isolated from fat without attachment to culture dishes (ADCs), or cells purified on the basis of cell surface markers for the osteoclastic precursors by methods such as fluorescent activated cell sorting (FACS) or magnetic bead separation.

Tissue Culture

Dulbecco's modified Eagle medium (DMEM, cat. no. 11320-033), 0.5% trypsin-EDTA (cat. no. 15400-054), and antibiotic-antimycotic (cat. no. 15240-062) were obtained from Invitrogen (Grand Island, N.Y.). Fetal bovine serum (FBS, cat. no. 2442) was from Sigma (St. Louis). Culture dishes were from NUNC Brand Products (Nalge Nunc, Rochester, N.Y.). Liberase Blendzyme 1 was from Roche (Roche cat. no. 1988417, Indianapolis, Ind.). Other methods for growing adipose cells or stem cells are described in U.S. Pat. No. 6,777,231, and U.S. Patent Publication Nos. 20050076396, 20040229351, 20040171146, 20040166096, 20040092011, 20030082152, 20020076400, and 20010033834, which are incorporated by reference herein in their entirety.

Serum-Free Culture

X-VIVO15™ and UltraCULTURE™ serum-free culture media (Biowhittaker) were both used. No additional growth factors or hormones were added, nor was serum or tissue extracts added as supplements.

Osteoclast Differentiation

Osteoclast differentiation was induced in vitro, in serum-containing or serum-free medium, supplemented with dexamethasone, M-CSF, and RANKL. Dexamethasone was used at 1×10⁻⁷ M, m-CSF at 25 ng/ml, and RANKL at 100 ng/ml.

A variety of osteoclast differentiation markers are available to assay osteoclast differentiation, several of which are used in the experiments disclosed herein or are known to those of ordinary skill in the art (see Katz et al., Stem Cells, 2005, 23:3:412-23; Blair et al., Biochem. Biophys. Res. Commun., 2005, 328:728-738; Quinn et al., Biochem. Biophys. Res. Commun., 2005, 328739-745; U.S. Pat. No. 6,861,257). Osteoclast differentiation was determined using several techniques. In some assays, differentiation was monitored by counting multinuclear cells, a marker for osteoclast differentiation. Differentiation was also assayed by identifying tartrate-resistant acid phosphatase (TRAP) cells. Other assays included immunostaining for the osteoclast markers calcitonin receptor, CD11b, and CD11c using monoclonal antibodies directed against these antigens.

Osteoblast Differentiation

To induce osteoblast differentiation, ASCs were cultured in the presence of β-glycerophosphate, dexamethasone, and ascorbic acid as previously described (Tholpady et al., Anat. Rec., 2003, 272:1:398-402).

### Example 1

### Osteoclastic Differentiation of ASCs In Vitro

Serum-Containing Medium—

Methods—

Human ADCs were plated on polystyrene tissue culture dishes or 8-well chamber glass slides at a density of 1×10⁵ cells per square centimeter. ADCs were cultured in media composed of DMEM/F12/10% FBS or DMEM/10% FBS in the presence or absence of 25 ng/mL macrophage colony-stimulating factor (M-CSF), 100 ng/mL recombinant human RANKL R&D Systems Inc., Minneapolis Minn., USA), and 1×10⁻⁷ M dexamethasone. Media was replaced every other day. After 32 days of culture, ASCs were fixed in 4% paraformaldehyde and stained with tartrate-resistant acid phophatase (TRAP), a specific marker.

Results

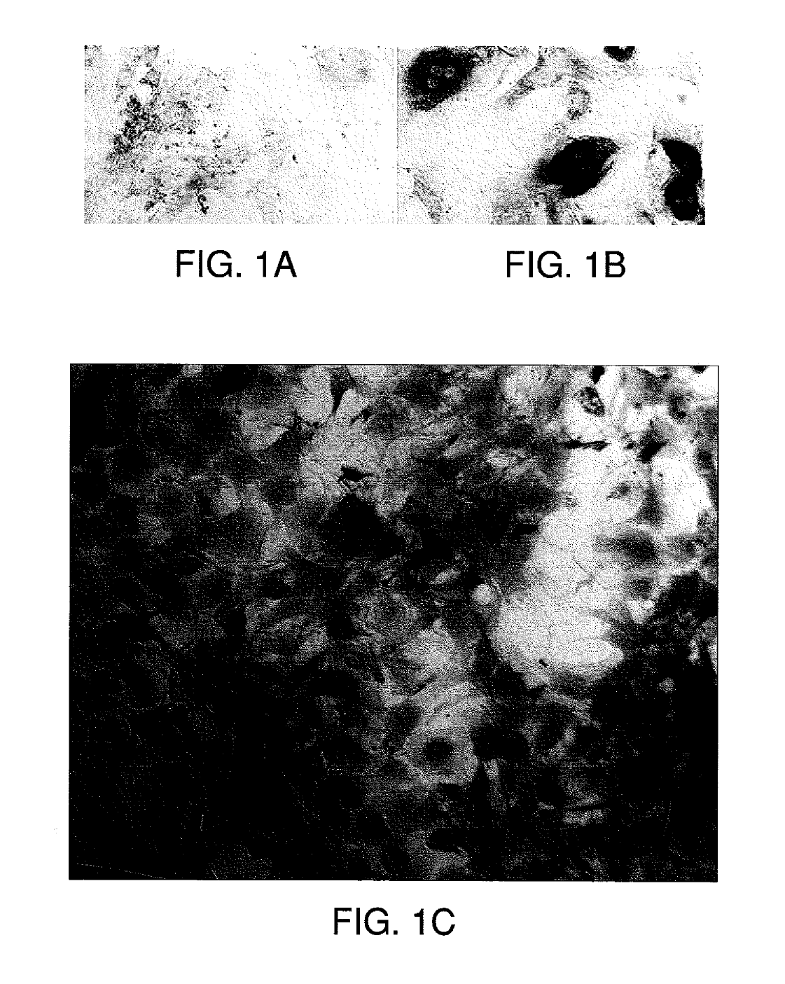

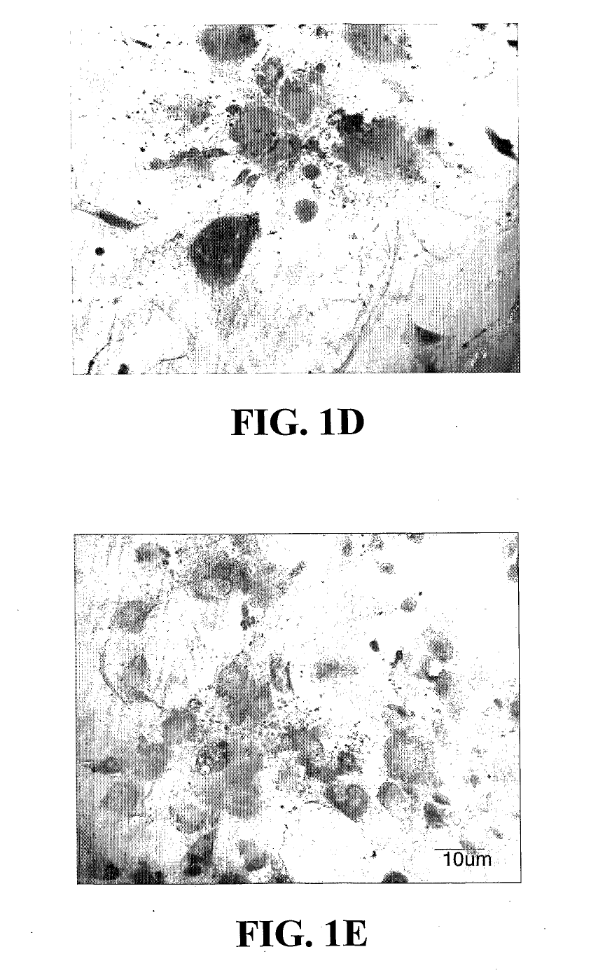

The presence of the dark brown TRAP positive cells, along with large multinucleated nuclei, confirmed the osteoclast phenotype (see FIG. 1).

Additional experiments in the presence of osteoclast differentiation-inducing medium indicated that at a seeding density of 10,000 ADCs in a 96 well plate, 20-50 multinucleated osteoclasts formed. Cells were assayed at 1, 2, and 3 weeks exposure to differentiation-inducing medium. Differentiation was assayed using the TRAP, multinucleation, calcitonin receptor, CD11b, and/or CD11c markers. Cells treated with the osteoclast differentiation media which had undergone multinucleation also exhibited actin rings around the nucleus. Even though there was not a high percentage of multinucleated cells in culture following treatment with osteoclast differentiation-inducing agents, the experiments demonstrated that most of the mononuclear cells had become TRAP positive after exposure to the differentiation-inducing medium (see FIG. 1C). The data suggest that longer exposure to the medium will result in the formation of more multinucleated osteoclasts. The data further suggest that the methods of isolating adipose tissue and the culture conditions used herein to establish populations of adipose tissue-derived cells are highly efficient with regard to yielding a population of adipose tissue-derived cells with the ability to differentiate into osteoclasts.

Serum-Free Media—

Methods

ADCs were tested for their growth and differentiation ability in serum-free media X-VIVO™ or UltraCULTURE™. Cells were induced to differentiate as described above. Differentiation was assayed as described above. In some experiments, tissue culture plates were precoated with calf skin collagen (“CSC”, Sigma) at 20 μg/cm².

Results

Growth of ADCs and osteoclastic differentiation of ASCs in X-VIVO15™ or UltraCULTURE™ serum-free media were comparable to media (DMEM/F-12) containing 10% FBS. While the cells were able to attach to tissue culture plastic in these media in 24 hours without the addition of serum, adding osteoclast differentiation components (1×10⁻⁷ M dexamethasone, 25 ng/ml m-CSF, and 100 ng/ml RANKL) caused cells to rapidly detach from the plates. Precoating the tissue culture plates with calf skin collagen (CSC, Sigma) at 20 μg/cm² promoted cell attachment for over 3 weeks while maintaining chemically defined, serum-free conditions for osteoclast differentiation. Even in serum-free conditions, the cells plated on collagen-coated dishes were able to proliferate and could be serially passaged. A high percentage of the cells were TRAP positive (FIG. 1C) Differentiation equivalent to that seen in medium with serum was also found in serum-free conditions (FIGS. 1D and 1E).

Summary—ADCs from six different individuals have been tested to date, using serum-containing or serum-free media. Similar results regarding growth and osteoclastic differentiation have been obtained for ADCs from all six individuals. Additionally, an Affymetrix array determined that M-CSF and RANKL MRNA were both present in the total ASC population.

It is known that adipose tissues contain more multipotent stem cells per unit than bone marrow or umbilical cord blood (Kern et al., 2006, Stem Cells, 24:1294-1301). The monocyte/macrophage precursors in peripheral blood that can form osteoclasts are committed progenitors rather than true stem cells. The concentration of monocyte precursors in peripheral blood ranges between 2,000,000-9,000,000 per liter in normal individuals. The process of isolating the cells would yield a lower number. The number of purified adherent ADCs from a typical preparation described herein is 2,000,000 per liter of lipoaspirate or kilogram of abdominal tissue. The exact number of osteoclast precursors present in the starting adipose tissues has not yet been determined, but the data demonstrate that far more stem cells are present in the adipose tissue specimens disclosed herein than in blood.

### Example 2

### Delivery of Human ASCs For Osteoclastic Differentiation In Vivo

Methods

Twelve retired breeder NCI nude rats were randomly assigned to two groups of six animals. Animals were anesthetized and hair over the calvaria was shaved and disinfected. Lidocaine was injected intradermally at the midline on top of the head. Rats were placed in a cephalostat and an incision was made at the midline. Subcutaneous fascia was separated from the periosteum and periosteal flaps were reflected laterally. A circular template with a diameter of 8-mm was marked on the skull just over the parietal bones. A dremel drill was used to drill an 8-mm defect in the skull. Saline irrigation was used to prevent bum death to adjacent bone and careful care was taken to prevent damage to underlying dura mater.

Type I collagen gels (5 mg/ml), with and without ASCs (1×10⁶ cells) which had been maintained in tissue culture between three and seven passages, were placed into defects. Periosteum was closed over the defect with 10-0 nylon suture, and skin was closed with 4-0 suture.

In some experiments, ASCs were treated with osteoblastic differentiation factors for 7, 10, or 14 days Katz et al., Anat. Rec., 2003, 272:1:398-402).

After 6 weeks, animals were euthanized and skulls were processed and embedded for immunohistochemistry. A human specific anti-mitochondria antibody (R & D Systems) was used to distinguish implanted human cells from rat cells in skull tissue. Additionally, sections were stained with hematoxylin and eosin.

Results

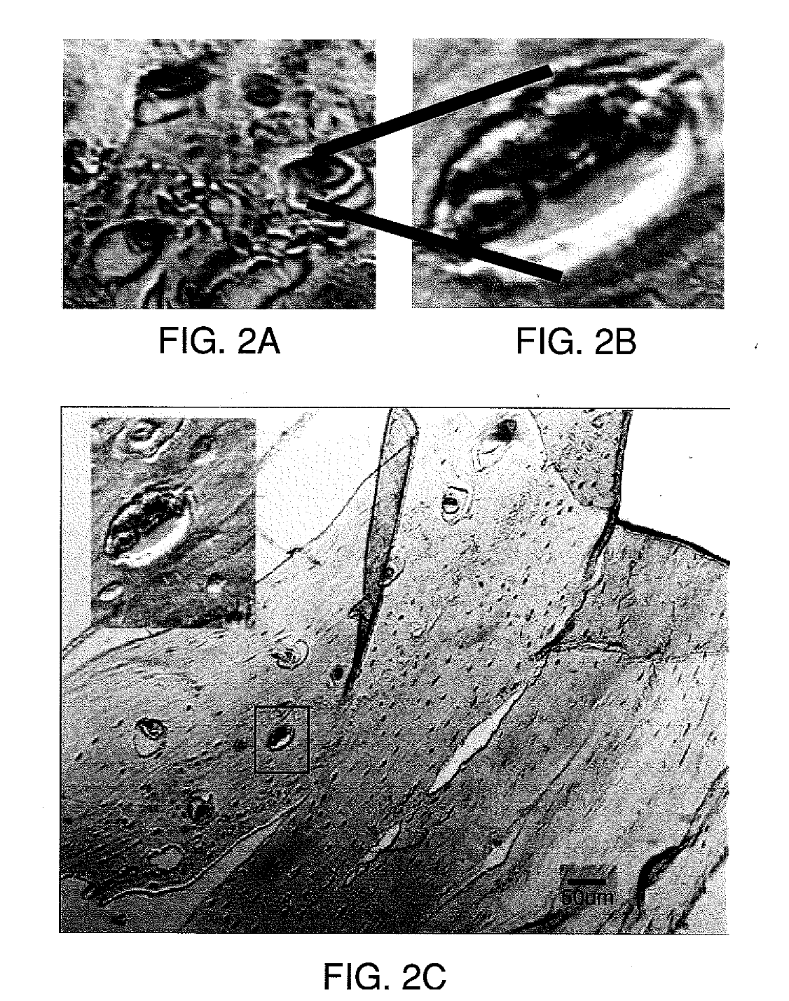

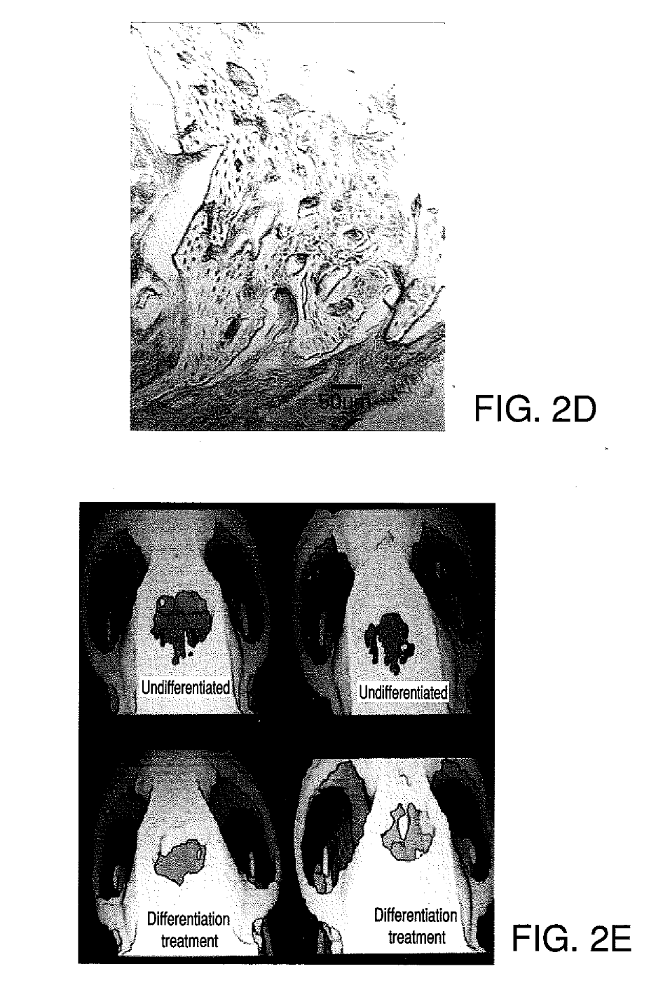

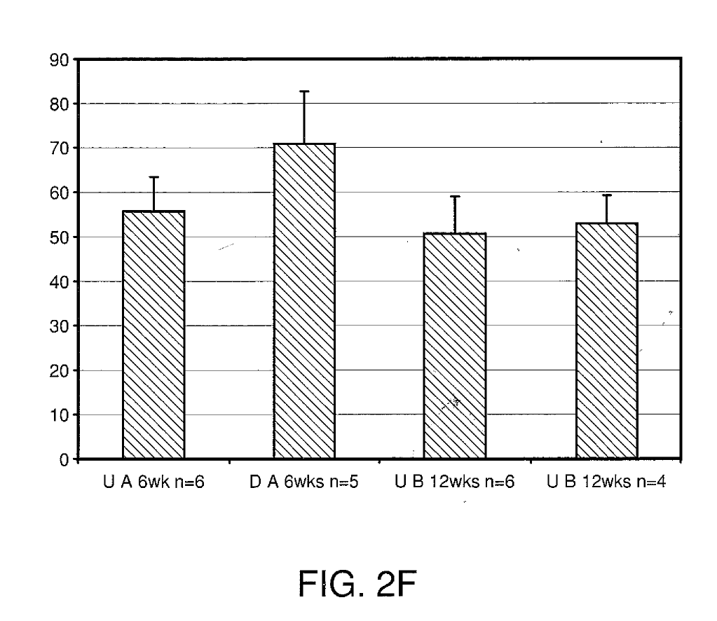

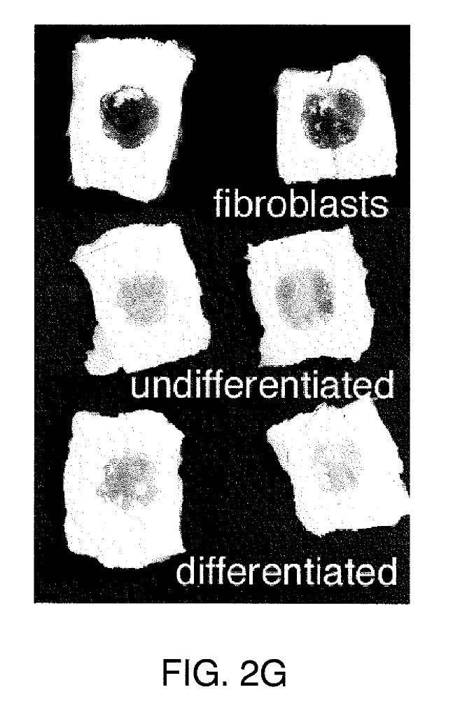

Microscopic examination revealed the presence of human multinucleated osteoclasts within resorption lacunae in the NIH-Nude rat bone (see FIG. 2). Positive staining (brown) with a human specific mitochondrial antibody (Chemicon) indicates that the osteoclasts are derived from the treated implanted human cells, rather than from the rat host (see FIG. 2). These human cells were also positive for calcitonin receptor. It was also found that cells which had been pretreated with osteoblast inducing factors prior to delivery to the cranial defect yielded greater cranial bone regeneration.

The images of 2A and 2B demonstrate the presence of multinucleated osteoclasts within resorption lacunae in the bone. Human cells are dark (brown), staining positively with a human specific mitochondrial antibody, indicating that the osteoclasts are derived from the implanted human cells rather than from the rat host. FIGS. 2C and 2D are low magnifications of the bone forming in the defect from a group treated with differentiated ASCs depicted in the histogram of FIG. 2F. In FIG. 2C, the tissue showed areas containing osteoclasts (at higher magnification in inset). The bone in this area shows well-organized lamellar bone of about 2 mm thickness. The osteoclast in the inset of FIG. 2C is within a resorption lacuna. The osteoclastic cells also stained positive for TRAP and calcitonin receptor (not shown). FIG. 2E represents 3D CT reconstructions of nude rats treated with ASCs in duragen collagen sponges in 8 mm defects. The ASCs were either treated for differentiation for 10 days or not treated, as indicated on the figure. ASCs promoted about 50% healing in 30 days. The undifferentiated ASCs appear to mediate greater bone remodeling and perhaps resorption, as seen in histology of FIGS. 2C and 2D. FIG. 2F represents a histogram of percentage of healing of an osseous defect (8 mm) in NIH-Nude rats (including the animals depicted in the X-rays images in right panel). FIG. 2G represents an image depicting contact planar X-ray images of cranial bone containing excised defects treated for 6 weeks with human skin fibroblasts (top panel), undifferentiated ASCs (middle panel), and osteoblast differentiated (10 days) ASCs delivered in collagen gels to the bone defect.

Summary

The groups depicted have been subjected to statistical analysis for significance. The trends appear to indicate differentiated cells regenerate bone better than undifferentiated ones from local delivery. It appears that undifferentiated cells retain developmental plasticity following local delivery—many finger-like projections of bone were observed throughout the healing site, which may represent remodeling activity, compared to the pattern of steady ingrowth of new bone from the margins of the defect seen with osteoblasts and more differentiated ASCs (FIG. 2). As seen in FIG. 2, some skulls of animals receiving undifferentiated cells have substantial areas of bone remodeling, which were not observed in several hundred rats receiving osteoblasts or fibroblasts, and these areas have many osteoclasts present that react with the anti-human mitochondria antibody.

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are hereby incorporated herein by reference in their entirety.

Other methods which were used but not described herein are well known and within the competence of one of ordinary skill in the art of clinical, chemical, cellular, histochemical, biochemical, molecular biology, microbiology and recombinant DNA techniques.

The description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein, but is to be accorded the widest scope consistent with the principles and novel features disclosed herein. The appended claims are intended to be construed to include all such embodiments and equivalent variations.

What is claimed is:

1. An isolated adipose tissue-derived cell, wherein said cell can differentiate into an osteoclast when contacted with at least one osteoclast differentiation-inducing compound.

2. The cell of claim 1, wherein said cell is a human cell.

3. A method of producing an osteoclast, said method comprising culturing an adipose tissue-derived cell in a culture medium comprising an effective concentration of at least one osteoclast differentiation-inducing compound, thereby producing an osteoclast.

4. The method of claim 3, wherein said osteoclast is a human osteoclast.

5. The method of claim 3, wherein said osteoclast differentiation-inducing compound is selected from the group consisting of dexamethasone, M-CSF, RANKL, TNF-α, TGF-β, IL-3, IL-7, GM-CSF, eotaxin, eotaxin-2, and eotaxin-3.

6. The method of claim 5, wherein said compound is selected from the group consisting of dexamethasone, M-CSF, and RANKL.

7. The method of claim 3, wherein said culture medium comprises serum.

8. The method of claim 3, wherein said culture medium is serum-free.

9. The method of claim 8, wherein said cells are cultured on tissue culture plates pretreated to enhance cell attachment.

10. The method of claim 9 wherein said tissue culture plates are pretreated with calf skin collagen.

11. The method of claim 8, wherein said serum-free medium is X-VIVO15™ or UltraCULTURE™.

12. A method of obtaining isolated osteoclast precursor cells, said method comprising obtaining adipose tissue from a subject, preparing said adipose tissue for cell culture, plating cells derived from said prepared adipose tissue into cell culture plates comprising growth medium, thereby obtaining isolated osteoclast precursor cells.

13. The method of claim 12, wherein said osteoclast precursor cell can differentiate into an osteoclast.

14. The method of claim 13, wherein said osteoclast expresses at least one osteoclast marker.

15. The method of claim 14, wherein said osteoclast marker is selected from the group consisting of TRAP, multinucleation, calcitonin receptor, CTSK, CD11b, and CD11c.

16. The method of claim 12, wherein said cell culture medium comprising serum.

17. The method of claim 16, wherein said cells proliferate in culture.

18. The method of claim 12, wherein said cell culture medium is serum-free.

19. The method of claim 18, wherein said serum-free cell culture medium is X-VIVO15™ or UltraCULTURE™.

20. The method of claim 18, wherein said cell culture plates are pretreated to enhance cell attachment.

21. The method of claim 20, wherein said cell culture plates are treated with calf skin collagen.

22. The method of claim 18, wherein said cells proliferate in culture.

23. A method of treating a subject with osteoclasts or osteoclast precursor cells, wherein said subject is suffering from a disease, disorder, or injury characterized by a bone defect, said method comprising the steps of:
a. obtaining adipose tissue comprising cells capable of differentiating into osteoclasts; and
b. administering to said subject a composition comprising an amount of said cells effective to treat said disease, disorder, condition or injury.

24. The method of claim 23, wherein said adipose tissue is cultured prior to administration to said subject.

25. The method of claim 23, wherein said disease, disorder, or injury is associated with aberrant osteoclast activity.

26. The method of claim 25, wherein said aberrant osteoclast activity is reduced osteoclast activity.

27. The method of claim 26, wherein said disease or disorder is selected from the group consisting of osteopetrosis, ADO II, dental abnormalities, pycnodysostosis, and cancer.

28. The method of claim 23, wherein said adipose tissue is obtained from said subject.

29. The method of claim 23, wherein said subject is human.

30. The method of claim 29, wherein said cells are pretreated with at least one osteoclast differentiation-inducing compound prior to administration to said subject.

31. The method of claim 23, wherein said cells are co-administered with adipose tissue-derived cells pretreated with at least one osteoblast differentiation-inducing compound.

32. The method of claim 23, wherein said cells are pretreated with at least one osteoclast differentiation-inducing compound prior to administration to said subject and are co-administered with adipose tissue-derived cells pretreated with at least one osteoblast differentiation-inducing compound.

33. The method of claim 23, wherein said composition comprises a delivery vehicle.

34. The method of claim 33, wherein said delivery vehicle is collagen gel.

35. The cell of claim 1, wherein said cell has been immortalized.

36. The cell of claim 1, wherein said cell has been modified to alter gene expression.

37. A method of identifying a compound which modulates osteoclast production, said method comprising contacting a culture comprising cells of claim 1 with a test compound and comparing the level of osteoclast production in said culture with the level of osteoclast production in an otherwise identical culture not contacted with said test compound, wherein a higher or lower level of said osteoclast production in said culture contacted with said test compound, compared with the level of osteoclast production in said culture not contacted with said test compound, is an indication that said test compound modulates osteoclast production, thereby identifying a compound which modulates osteoclast production.

38. The method of claim 37, wherein said compound inhibits osteoclast production.

39. The method of claim 37, wherein said compound stimulates osteoclast production.

40. A compound identified by the method of claim 37.

41. A method of identifying a compound which modulates osteoclast activity, said method comprising inducing cells of claim 1 to differentiate into osteoclasts in culture, contacting a culture comprising said osteoclasts with a test compound and comparing the level of osteoclast activity in said culture with the level of osteoclast activity in an otherwise identical culture not contacted with said test compound, wherein a higher or lower level of said osteoclast activity in said culture contacted with said test compound, compared with the level of osteoclast activity in said culture not contacted with said test compound, is an indication that said test compound modulates osteoclast activity, thereby identifying a compound which modulates osteoclast activity.

42. The method of claim 41, wherein said compound inhibits osteoclast activity.

43. The method of claim 41, wherein said compound stimulates osteoclast activity.

44. A compound identified by the method of claim 41.

45. A method of treating a subject suffering from a disease, disorder, or injury characterized by a bone defect, said method comprising administering a therapeutically-effective amount of compound identified according to the method of claim 37.

46. A method of treating a subject suffering from a disease, disorder, or injury characterized by a bone defect, said method comprising administering a therapeutically-effective amount of a compound identified according to the method of claim 41.

47. A method of generating an implantable graft for use in treating a subject suffering from a disease, disorder, or injury characterized by a bone defect, wherein said method comprises combining a cell of claim 1 with a cellular or non-cellular material.
